(19) United States
(12) Patent Application Publication
(10) Pub. No.: US 2006/0057230 A1
(43) Pub. Date: Mar. 16, 2006

(54) Tea polyphenols bioavailability enhancement method

(76) Inventor: Hsiao-Hui Sherry Chow, Tucson, AZ (US)

Correspondence Address: QUARLES & BRADY STREICH LANG, LLP, ONE SOUTH CHURCH AVENUE, SUITE 1700, TUCSON, AZ, 85701-1621, US

(21) Appl. No.: 11/224,427

(22) Filed: Sep. 12, 2005

Related U.S. Application Data
(60) Provisional application No. 60/608,648, filed on Sep. 10, 2004.

(51) Int. Cl. A61K 36/82

(52) U.S. Cl. 424/729

(57) ABSTRACT

Methods for increasing tea polyphenol bioavailability. Dosing conditions, such as fasting for defined periods, result in greater oral bioavailability of free catechins.

### CROSS-REFERENCE TO RELATED APPLICATIONS

This application claims the benefit of priority to U.S. Provisional Application No. 60/608,648 entitled “Enhancing Oral Bioavailability of Tea Polyphenols by Taking Tea Polyphenols Under the Fasting Condition” filed on Sep. 10, 2004, the entire contents of which are incorporated by reference.

### U.S. GOVERNMENT RIGHTS

This invention was made with Federal Government support under Contract Number N01-CN-25119 awarded by the National Cancer Institute. The Federal Government has certain rights in the invention.

### BACKGROUND OF THE INVENTION

1. Field of the Invention

The invention relates generally to preventative health methods and more particularly to methods that enhance the bioavailability of tea polyphenols.

2. Description of the Related Art

Tea (Camellia sinensis) is one of the most consumed beverages in the world, especially in Asian countries. The relationship between tea consumption and cancer has been a subject of research interest for many investigators in the past decade. Indeed, several recent publications have thoroughly reviewed and summarized epidemiological and experimental studies on tea and cancer prevention.

The abbreviations used hereinafter are as follows: EGCG, epigallocatechin gallate; EGC, epigallocatechin; EC, epicatechin; ECG, epicatechin gallate; CL/F, oral clearance; Vd/F, oral apparent volume of distribution; AUC, area under the plasma concentration-time curve.

Because the highly polymerized components in black tea are not well characterized, experimental studies demonstrating the chemopreventive effects of tea have been conducted primarily with green tea. The evidence obtained in vitro and from animal studies in vivo concerning potentially protective effects of green tea or green tea components is compelling. Green tea, green tea extract, green tea polyphenols, and epigallocatechin gallate (EGCG, a major green tea component) have been shown to inhibit carcinogenesis induced by a wide variety of carcinogens in rodent cancer models. Moreover, cancer chemopreventive activity has been demonstrated in the following target organs: colon, duodenum, esophagus, forestomach, large intestine, liver, lung, mammary glands, and skin.

The anticarcinogenic properties of green tea have been suggested to be due, in part, to the antioxidant effect of green tea catechins. Studies also have suggested that the cancer preventive properties of tea are related to inhibition of tumor promotion and cell proliferation. An additional pharmacological property of green tea catechins is their potentials in modulating carcinogen metabolizing enzymes, leading to reduced carcinogen-induced DNA damage.

The epidemiological evidence on the protective effect of green tea consumption against the development of human cancers is not conclusive. Some studies suggested that green tea consumption may reduce the risk of certain cancers; such a protective effect has not been observed in other studies. The inconsistent epidemiological findings may be attributed to compounding variables, such as individualized differences in tea preparation and consumption patterns, variability associated with tea production, variability in the bioavailability of the active green tea constituents, concomitant use of tobacco and alcohol, and individualized differences in life style.

The pharmacokinetics of green tea catechins following single and multiple dose administration of a defined green tea catechin extract (Polyphenon E) and EGCG has recently been determined. The oral bioavailability of tea catechins was found to be low, resulting in plasma concentrations 5-50 times less than concentrations shown to exert biological activities in in vitro systems. A number of factors may affect the oral bioavailability of green tea catechins and subsequently their biological responses. Conversely, in a small pilot study, free EGCG plasma concentration determined at a single time point (90 min post-dose) after ingestion of 3, 5, or 7 capsules of Sunphenon DCF-1 (corresponding to 225, 375, and 525 mg EGCG, respectively) was 300, 1970, and 2020 ng/ml, respectively. These levels were significantly higher than those reported in previous investigations.

Thus, there continues to be a need to evaluate the chemopreventive activity of ingestion of green tea or green tea components and to develop methods for enhancing bioavailability of tea components.

### SUMMARY OF THE INVENTION

The invention relates in general to a method for administering a tea polyphenol that includes one or more dosing conditions. The green tea polyphenols include epigallocatechin gallate (EGCG), epigallocatechin, epicatechin gallate, and epicatechin.

In one aspect of the invention, the dosing condition includes the step of providing instructions to a user to fast for a defined period prior to tea polyphenol intake. Preferably, instructions are in printed or electronic form.

In another aspect of the invention, a method is provided for increasing catechins in blood serum. Preferably, the method includes the steps of fasting for a defined period and consuming a tea polyphenol after that defined period, whereby catechins increase in concentration in blood serum by at least two fold.

In yet another aspect of the invention, a method is provided for enhancing chemoprevention that includes the step of a user consuming a tea polyphenol after fasting for at least 6 hours. Preferably, this method further includes the step of fasting after tea polyphenol intake for at least 5 minutes and most preferably for at least 1 hour.

In still another aspect of the invention, the tea polyphenol provided includes between 400 and 1,200 milligrams of Polyphenon E. Preferably, the fasting instructions provided should apply to Polyphenon E does of 800 milligrams of less (based on EGCG content) due to better gastrointestinal tolerability.

Various other purposes and advantages of the invention will become clear from its description in the specification that follows. Therefore, to the accomplishment of the objectives described above, this invention includes the features hereinafter fully described in the detailed description of the preferred embodiments, and particularly pointed out in the claims. However, such description discloses only some of the various ways in which the invention may be practiced.

### BRIEF DESCRIPTION OF THE DRAWINGS

FIG. 1A depicts the average plasma free EGCG concentration versus time profiles after oral administration of Polyphenon E (400 mg EGCG) in the absence or presence of food. Each point represents the mean data from 10 subjects, bars, ±SE.

FIG. 1B depicts the average plasma free EGCG concentration versus time profiles after oral administration of Polyphenon E (800 mg EGCG) in the absence or presence of food. Each point represents the mean of data from 10 subjects, bars, ±SE.

FIG. 1C depicts the average plasma free EGCG concentration versus time profiles after oral administration of Polyphenon E (1200 mg EGCG) in the absence or presence of food. Each point represents the mean of data from 10 subjects, bars, ±SE.

### DETAILED DESCRIPTION OF THE PREFERRED EMBODIMENTS

The invention generally relates to a method for enhancing green tea polyphenol bioavailability through providing dosing conditions involving fasting. The oral bioavailability of the major green tea constituents, green tea catechins, is low, resulting in systemic catechin levels many folds less than the effective concentrations determined in in vitro systems. Thus, the invention involves providing dosing conditions that increase polyphenol biovailability through defined periods of fasting.

As used herein, the term “fasting” means avoiding substantially all food and beverage (except water) intake.

The invention involves providing instructions to a user to fast for a defined period of time, preferably for at least 6 hours prior to and for at least 5 minutes (and more preferably at least 1 hour after) taking a tea polyphenol.

The instructions may take the form of printed (e.g., product packaging or a brochure) or electronic matter (e.g., an Internet homepage) and may be provided separate from or together with a tea polyphenol.

Tea polyphenols are available in many forms for consumption, including capsule and liquid form (e.g., hot tea beverages). Thus, the benefits of polyphenols are conveniently available for enhancement through the dosing condition of the present invention.

The antioxidant and other healthful effects of tea polyphenol intake depend on the polyphenol concentration in the blood. Thus, the invention provides a method for increasing catechins in blood serum that includes fasting for a defined period and consuming a tea polyphenol after the defined period of fasting has ended. Again, this method preferably involves an additional fasting step, i.e., fasting for at least 5 minutes (and preferably 1 hour) after the tea polyphenol is consumed.

Repeated consumption of tea polyphenols is thought to result in a chemopreventive effect. Thus, the invention provides a new and improved method for enhancing tea polyphenol chemopreventive activities through a user consuming a tea polyphenol after fasting for at least 6 hours. Accordingly, a tea polyphenol may be consumed with regularity, such as once a day before a morning meal or before more than one meal a day.

The chemopreventive (e.g., antioxidant and other healthful effects) of tea polyphenols are thought to result from increased concentration of tea components in a user's bloodstream. Therefore, the fasting step of the invention provides for an increase in a user's blood serum concentration of a catechin by at least two fold. Moreover, the level of a catechin in a polyphenol user's blood may remain elevated longer by fasting for at least 5 minutes after tea consumption, with fasting of at least 1 hour being most preferred.

When Polyphenon E is the tea polyphenol consumed, a dose of 800 milligrams or less (based on EGCG content) is preferred in order to minimize stomach or other irritations in some users.

The following non-limiting example is illustrative of the invention.

Study Drugs. Polyphenon E capsules were supplied by the Chemoprevention Agent Development Research Group, National Cancer Institute (Bethesda, Md.). Each capsule contained 416.7 mg Polyphenon E (200 mg EGCG, 48.5 mg EGC, 34.2 mg EC, 20 mg ECG, and other tea catechins), 28.8 mg pregelatinized starch, 2.25 mg colloidal silicon dioxide, and 2.25 mg magnesium stearate in size 0 gelatin capsules. The study medications were stored at room temperature and protected from environmental extremes.

Participants. Thirty non-smoking healthy men and women ≧18 years of age participated in the study. The participants have normal liver and renal function. Participants were excluded if they were pregnant, had cancers of any type within the past 5 years, had severe metabolic disorders or other serious acute or chronic diseases, had consume tea regularly, and had participated in other clinical research studies within the past 3 months. The study was approved by the University of Arizona Human Subjects Committee. Written informed consent was obtained from all participants.

Study Design. During the initial clinic visit, study participants completed a medical history form and underwent a brief physical examination. A fasting blood was collected and subjected to a complete blood count with differential leukocyte count and a comprehensive blood chemistry analysis. Eligible subjects were randomly assigned to one of the Polyphenon E doses (400, 800, and 1200 mg, based on EGCG content) and were required to refrain from the ingestion of tea, apples, chocolate, and their products two weeks prior to the first pharmacokinetic study day and until the end of the second pharmacokinetic study day. The night prior to the first pharmacokinetic study day, subjects fasted after midnight except for water. At approximately either 6 am or 9 am on the morning of the first pharmacokinetic study day, each subject was randomly assigned to receive Polyphenon E at the assigned dose level with or without breakfast. Breakfast consisted of one or two 4-oz muffins.

Study participants had the option of selecting from three varieties of Otis Spunkmeyer® brand muffins: cheese streusel, wild blueberry, and banana nut flavors. The muffins contain 420 to 480 calories each, with 48-60 grams of carbohydrates, 20-24 grams of fat, and 6 grams of protein. Blood samples were collected prior to and at 0.5, 1, 2, 4, 6, 8, 10, and 24 hours after Polyphenon E administration. Urine samples were collected prior to and up to 24 hours (divided into two intervals: 0-8 and 8-24 hours post-dose) after dosing. Following a one-week wash-out period, study subjects fasted overnight and were crossed-over to receive the same dose of Polyphenon E under the other fasting/fed condition.

Sample Collection and Processing. Blood samples were collected into Vacutainer tubes containing sodium heparin. Within 30 minutes of collection, tubes were centrifuged for 10 minutes at 2000 rpm. After centrifugation, plasma was mixed with Vc-EDTA solution (0.4 M NaH₂PO₄ buffer containing 20% ascorbic acid and 0.1% EDTA, pH 3.6) in the volume ratio of 1:0.02 and stored at −80° C. until sample analysis. Urine specimen container was preadded with 1.38 g NaH₂PO₄, 1 g ascorbic acid, and 5 mg EDTA to prevent degradation of tea catechins. Urine specimen was kept cold by storing the specimen container in a cooler chest with freezer packs during each collection period. The total volume of each urine sample was measured. An aliquot of urine sample was mixed with the Vc-EDTA solution and stored at −80° C. until sample analysis.

Tea Polyphenol Concentration Measurements. EGCG, EC, EGC, ECG concentrations in plasma and urine samples were determined using a published method with minor modifications. In brief, for determination of free green tea catechins, plasma or urine samples were extracted with ethyl acetate. The ethyl acetate layer was mixed with a small aliquot of 0.1% ascorbic acid before drying by vacuum centrifugation. The dried residue was re-dissolved in 15% acetonitrile and injected onto HPLC. For determination of the total of free and glucuronic acid/sulfate conjugates of tea catechins, plasma or urine samples were mixed with an aliquot of β-glucuronidase and sulfatase in the presence of ascorbate-EDTA solution. Following pre-treatment, the samples were extracted as described above for the free catechins.

The HPLC system consisted of an ESA Model 465 refrigerated autosampler, an ESA Model 580 two-pump solvent delivery system, an ESA 5500 coulochem electrode array system (CEAS), and a Supelcosil C₁₈ reversed-phase column (150×4.6 mm; particle size, 5 μm; Supelco Inc., Bellefonte, Pa.). The autosampler and column temperatures were maintained at 6° C. and 35° C., respectively. This assay employed a gradient of two mobile phases. Buffer A consisted of 30 mM NaH₂PO₄ buffer, acetonitrile, and tetrahydrofuran in the volume ratio of 98.13:1.75:0.12 (pH 3.35). Buffer B consisted of 15 mM NaH₂PO₄ buffer, acetonitrile, and tetrahydrofuran in the volume ratio of 41.5:58.5:12.5 (pH 3.45). The flow rate was maintained at 1 ml/min. The column was eluted with 96% buffer A and 4% buffer B from 0 to 7 min. Then the linear gradient was changed progressively to 17% buffer B at 25 min; 28% at 31 min, 33% at 37 min; 98% at 38 min. It was maintained at 98% from 38 to 43 min and finally changed back to 4% buffer B at 44 min for the analysis of the next sample. The eluent was monitored by the CEAS with potential settings at −10, 150, 300, and 500 mV.

Data Analysis. The following pharmacokinetic parameters of free EGCG were estimated using the WINNONLIN program (version 4.0.1) with the non-compartment approach: Tmax (time to reach the maximum plasma drug concentration), Cmax (maximum plasma drug concentration), AUC (area under the plasma drug concentration-time profile), CL/F (systemic clearance/bioavailability), Vd/F (apparent volume of distribution/bioavailability), and t½ (elimination half-life). The Cmax of free EGC, EC, and ECG, and of total catechins (free and conjugated) was obtained by visual inspection of the concentration-time data.

The amount of total EGC and EC excreted in the urine for each collection interval was calculated by the product of urinary catechin concentration and urine volume. The total amount excreted over 24 hours after dosing was obtained by adding the amount excreted over 0-8 hours and 8-24 hours after dosing.

The distribution of pharmacokinetic parameter data was normalized by logarithmic transformation prior to statistical analyses. The primary analyses were to determine whether the pharmacokinetics of tea catechins are different under fasting/fed states. Pharmacokinetic parameters such as AUC, Cmax, Tmax, half-life, CL/F, Vd/F, and total amount excreted over 24 hr between the fasting and fed conditions were compared by standard cross-over statistical analyses. The pkcross routine in Stata 8.0 (StataCorp 2003) was used. In this software, the default parameterization estimates overall mean, period effects, treatment effects, and sequence effects, assuming no carryover effects. A p<0.05 was considered statistically significant. In secondary analysis, EGCG pharmacokinetic parameters such as CL/F, Vd/F, dose normalized AUC, and dose normalized Cmax were compared among different dose levels using one-way analysis of variance followed by post-hoc t-tests corrected for multiple comparisons. For the post-hoc t-test, a p<0.0167 was considered statistically significant.

Results. Table 1 summarizes the demographic data of the study participants.

TABLE 1
Subject Demographic Data by Dose Levels.

| | 400 mg | 800 mg | 1200 mg |
|---|---|---|---|
| Number of subjects | 10 | 10 | 10 |
| Number of Male | 4 | 2 | 1 |
| Mean age in yrs | 42 (20-56) | 39 (21-51) | 45 (22-66) |
| (range) | | | |
| Height (in)* | 67 ± 3 | 66 ± 4 | 64 ± 5 |
| Weight (lbs)* | 165 ± 45 | 179 ± 52 | 157 ± 46 |

*mean ± one standard deviation

A total of 30 subjects (10/dose group) completed the study. There were no significant differences in the average age, weight, and height among dose groups. There were between 1 to 4 male participants in each treatment group.

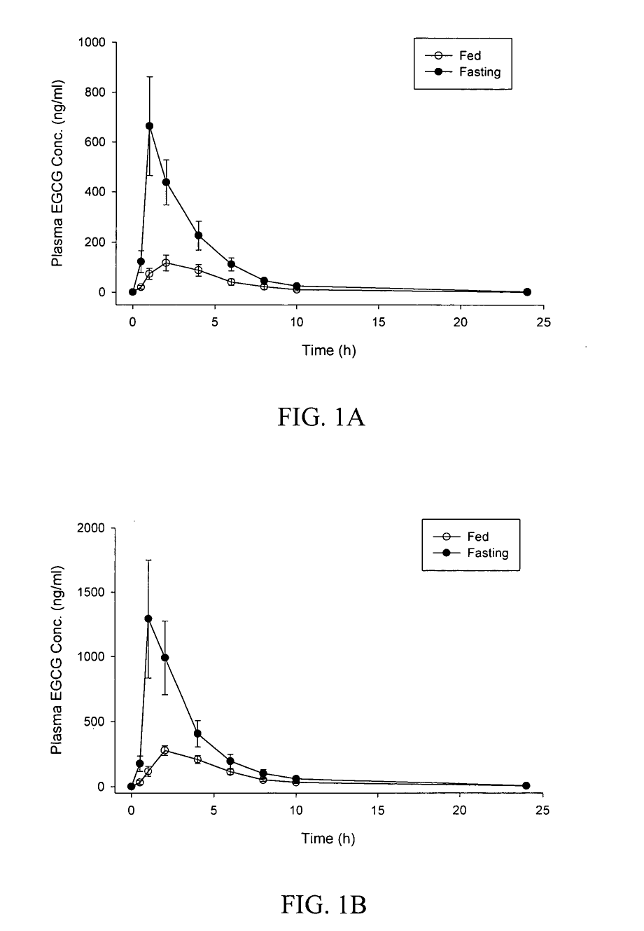

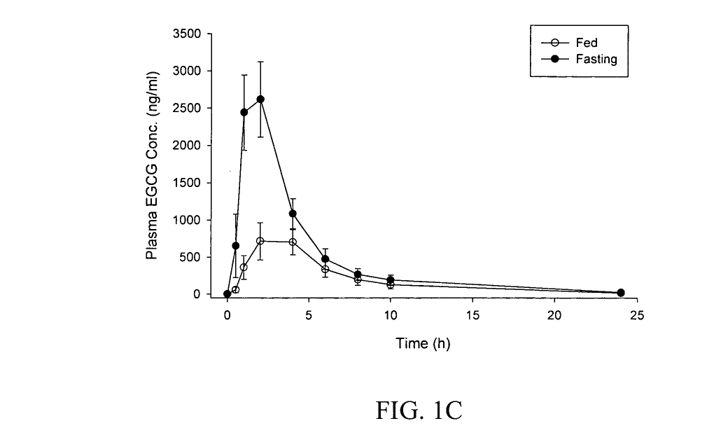

FIGS. 1A-1C illustrate the average plasma free EGCG concentration-time profiles after Polyphenon E administration in the absence or presence of food. After dosing, plasma EGCG levels increased toward a peak and declined rapidly as a function of time. A significant increase in plasma EGCG levels was noted when Polyphenon E was taken on an empty stomach following an overnight fast in comparison with that taken with food. The effect of dosing condition on plasma EGCG levels is consistently observed across all three dose levels.

Table 2 summarizes the average pharmacokinetic parameters of free EGCG after taking Polyphenon E on an empty stomach or with food.

TABLE 2
Pharmacokinetic parameters of free EGCG following oral administration of
Polyphenon E.

| PK | 400 mg | | 800 mg | | 200 mg | |
|---|---|---|---|---|---|---|
| Parameter | Fed | Fasting | Fed | Fasting | Fed | Fasting |
| AUC | 36.70 ± 24.66¹ | 126.96 ± 47.02² | 90.89 ± 37.39 | 254.48 ± 214.21² | 299.40 ± 285.86² | 685.53 ± 394.33² |
| (min · μg/ml) | | | | | | |
| Cmax | 141.8 ± 89.1 | 798.7 ± 573.1² | 294.0 ± 113.5 | 1522.4 ± 1357.8² | 923.6 ± 755.3 | 3371.6 ± 1651.2² |
| (ng/ml) | | | | | | |
| Tmax | 122.90 ± 83.70 | 93.90 ± 58.99³ | 154.90 ± 78.31 | 83.30 ± 31.04³ | 175.10 ± 74.58 | 90.60 ± 28.37³ |
| (min) | | | | | | |
| CL/F | 14.53 ± 7.84 | 3.64 ± 1.55² | 11.63 ± 9.25 | 4.80 ± 2.69² | 7.39 ± 4.86 | 2.37 ± 1.32² |
| (L/min) | | | | | | |
| Vd/F | 2872.8 ± 2672.8 | 912.4 ± 695.6² | 3112.9 ± 2810.8 | 946.6 ± 558.3² | 2825.1 ± 2257.7 | 664.0 ± 223.5² |
| (L) | | | | | | |
| t_{1/2} | 145.2 ± 129.1 | 170.5 ± 104.6 | 220.9 ± 209.3 | 156.5 ± 77.5 | 254.9 ± 59.9⁴ | 228.4 ± 75.3 |
| (min) | | | | | | |

¹mean ± one standard deviation.
²significantly different from the fed condition when data were pooled from all doses, p < 0.0001.
³significantly different from the fed condition when data were pooled from all doses, p < 0.0005.
⁴significantly different from 400 mg dose of the same dosing condition, p < 0.005.

The EGCG AUC obtained without food was significantly higher than that obtained with food for all three doses (126.96±47.02 vs. 36.70±24.66 min·μg/ml at 400 mg dose level; 254.48±214.21 vs. 90.89±37.39 min·μg/ml at 800 mg dose level; 685.53±394.33 vs. 299.40±285.86 min·μg/ml at 1200 mg dose level, p<0.0001 when data were pooled from all three doses). Similarly, the EGCG Cmax obtained without food was significantly higher than that obtained with food (798.7±573.1 vs. 141.8±89.1 ng/ml at 400 mg dose level; 1522.4±1357.8 vs. 294.0±113.5 ng/ml at 800 mg dose level; 3371.6±1651.2 vs. 923.6±755.3 ng/ml at 1200 mg dose level; p<0.0001 when data were pooled from all three doses). The dosing condition also has a significant effect on oral clearance (CL/F) and apparent volume of distribution (Vd/F), mostly due to an increase in the oral bioavailability (F) of EGCG in the fasting condition.

Table 3 summarizes the maximum plasma concentrations of free catechins after oral administration of Polyphenon E under the fasting and fed states.

TABLE 3
Maximum plasma free catechin concentrations (ng/ml) following an oral
administration of Polyphenon E.

| | Polyphenon E | | Polyphenon E | | Polyphenon E | |
|---|---|---|---|---|---|---|
| | (400 mg EGCG) | | (800 mg EGCG) | | (1200 mg EGCG) | |
| Catechin | Fed | Fasting | Fed | Fasting | Fed | Fasting |
| EGC | 15.9 ± 16.4¹ | 39.7 ± 44.6² | 34.1 ± 20.3 | 74.3 ± 70.0² | 42.9 ± 36.7 | 131.0 ± 94.2² |
| EC | 1.6 ± 3.9 | 5.6 ± 7.8 | 2.8 ± 5.0 | 1.92 ± 3.4 | 10.1 ± 15.3 | 16.6 ± 30.0 |
| EGCG | 141.8 ± 89.1 | 798.7 ± 573.1³ | 294.0 ± 113.5 | 1522.4 ± 1357.8³ | 923.6 ± 755.3 | 3371.6 ± 1651.2³ |
| ECG | 15.7 ± 8.8 | 87.6 ± 62.0³ | 33.9 ± 11.0 | 174.4 ± 141.1³ | 112.9 ± 88.2 | 382.6 ± 176.0³ |

¹mean ± one standard deviation.
²significantly different from the fed condition when data were pooled from all doses, p < 0.001.
³significantly different from the fed condition when data were pooled from all doses, p < 0.0001.

As observed in our previous studies, free EC levels are very low or undetectable in plasma samples. Similar to changes observed for plasma free EGCG, plasma free EGC and ECG Cmax levels in the fasting condition were significantly higher than those in the fed condition.

Table 4 summarizes the maximum plasma concentrations of total catechins after oral administration of Polyphenon E in the absence or presence of food.

TABLE 4
Maximum plasma total catechin concentrations (ng/ml)
following an oral administration of Polyphenon E.

| | | |
|---|---|---|
| Polyphenon E | Polyphenon E | Polyphenon E |
| (400 mg EGCG) | (800 mg EGCG) | (1200 mg EGCG) |

| Catechin | Fed | Fasting | Fed | Fasting | Fed | Fasting |
|---|---|---|---|---|---|---|
| EGC | 128.6 ± 74.9¹ | 81.8 ± 67.3 | 177.1 ± 86.2 | 135.5 ± 83.6 | 200.3 ± 115.1 | 323.4 ± 231.1 |
| EC | 156.3 ± 98.1 | 94.7 ± 63.3² | 234.0 ± 122.7 | 149.8 ± 79.7² | 346.2 ± 218.0 | 272.3 ± 173.6² |
| EGCG | 174.4 ± 80.6 | 754.9 ± 399.9³ | 360.1 ± 136.9 | 1622.7 ± 1501.4³ | 871.9 ± 625.3 | 3988.4 ± 2068.2³ |
| ECG | 21.2 ± 9.0 | 86.3 ± 48.8³ | 48.7 ± 17.5 | 197.5 ± 179.3³ | 117.5 ± 91.5 | 450.7 ± 218.7³ |

¹mean ± one standard deviation.
²significantly different from the fed condition when data were pooled from all doses, p < 0.005.
³significantly different from the fed condition when data were pooled from all doses, p < 0.0001.

Total catechin levels are determined after plasma samples have been treated with β-glucuronidase and sulfatase. Differences between total and free catechin levels represent the concentrations of conjugated catechins. Comparing the total and free catechin Cmax data suggests that EGC and EC are mostly present in plasma as the conjugated forms and EGCG and ECG are mostly present as the free form. The fasting effect on the Cmax of total EGCG and ECG is consistent with that of free EGCG and ECG. Taking Polyphenon E under the fasting state did not have a significant effect on the plasma levels of total EGC, but resulted in significantly lower plasma levels of total EC (p<0.005).

The urine samples collected for this study have been analyzed for total catechin levels after subjecting the samples to glucuronidase and sulfatase treatment. Similar to what was observed previously, urinary EGCG and ECG levels were very low or undetectable following Polyphenon E administration. Table 5 summarizes the amount of total EGC and EC recovered in the urine over 24 hours following oral administration of Polyphenon E.

TABLE 5
Amounts of total catechin (mg) recovered in the urine over 24 hours
following an oral administration of Polyphenon E.

| | Polyphenon E | | Polyphenon E | | Polyphenon E | |
|---|---|---|---|---|---|---|
| | (400 mg EGCG) | | (800 mg EGCG) | | (1200 mg EGCG) | |
| Catechin | Fed | Fasting | Fed | Fasting | Fed | Fasting |
| EGC | 4.85 ± 1.83¹ | 2.67 ± 1.25² | 5.54 ± 1.59 | 4.49 ± 3.15² | 6.48 ± 4.10 | 5.39 ± 3.09² |
| EC | 4.62 ± 1.76 | 2.64 ± 1.13³ | 6.49 ± 2.28 | 5.26 ± 3.54³ | 9.20 ± 6.25 | 6.44 ± 4.11³ |

¹mean ± one standard deviation.
²significantly different from the fed condition when data were pooled from all doses, p < 0.005.
³significantly different from the fed condition when data were pooled from all doses, p < 0.0005.

Taking Polyphenon E on an empty stomach resulted in less amounts of total EGC and EC recovered in the urine than those recovered after taking Polyphenon E with food.

Table 6 lists adverse events deemed possibly or probably related to study agent because of temporal proximity.

TABLE 6
Summary of incidence of reported adverse events possibly or probably related to
Polyphenon E administration, occurring within 24 hours of Polyphenon dose.

| | | Dose and Dosing Condition | | | | | |
|---|---|---|---|---|---|---|---|
| | | 400 mg | | 800 mg | | 1200 mg | |
| | | Fed | Fasting | Fed | Fasting | Fed | Fasting |
| Side effect | NCI grade | (n = 10) | (n = 10) | (n = 10) | (n = 10) | (n = 10) | (n = 10) |

Gastrointestinal

| | | | | | | | |
|---|---|---|---|---|---|---|---|
| Nausea | 1 | 0 | 0 | 2 | 1 | 2 | 7 |
| | 2 | 0 | 1 | 0 | 1 | 1 | 1 |
| Dyspepsia | 1 | 0 | 0 | 1 | 1 | 1 | 0 |
| | 2 | 0 | 0 | 0 | 0 | 0 | 0 |
| Diarrhea | 1 | 0 | 0 | 0 | 1 | 0 | 1 |
| | 2 | 0 | 0 | 0 | 0 | 0 | 0 |
| Other (gas, | 1 | 1 | 0 | 0 | 0 | 0 | 1 |
| eructation) | 2 | 0 | 0 | 0 | 0 | 0 | 0 |

Pain

| | | | | | | | |
|---|---|---|---|---|---|---|---|
| Abdominal pain | 1 | 1 | 0 | 0 | 0 | 0 | 2 |
| | 2 | 0 | 0 | 0 | 1 | 1 | 0 |
| Headache | 1 | 0 | 1 | 2 | 2 | 1 | 0 |
| | 2 | 0 | 0 | 0 | 0 | 0 | 0 |

Neurology

| | | | | | | | |
|---|---|---|---|---|---|---|---|
| Dizziness | 1 | 0 | 0 | 1 | 0 | 0 | 0 |
| | 2 | 0 | 0 | 0 | 0 | 0 | 0 |

Dermatology

| | | | | | | | |
|---|---|---|---|---|---|---|---|
| Rash | 1 | 0 | 0 | 0 | 0 | 0 | 0 |
| | 2 | 0 | 0 | 0 | 0 | 0 | 1 |

All doses of Polyphenon E used in this trial were generally well tolerated. The incidence of gastrointestinal adverse events, particularly nausea, increased at the higher doses and under fasting conditions. The headaches experienced by a number of participants were felt to be related to absence of habitual caffeine beverage consumption on the long pharmacokinetic study days.

The oral bioavailability of tea catechins has been found to be low in rodents. It has been reported that less than 2% of EGCG dose administered orally was available in the systemic blood in rats. An oral bioavailability of <13% was recently reported for EGCG in mice. The oral bioavailability of tea catechins has not been determined in humans because of the lack intravenous formulations. We have recently determined the pharmacokinetics of green tea catechins following single and multiple dose administration of Polyphenon E and EGCG. The oral clearance (CL/F) and apparent volume of distribution (Vd/F) were found to be around 6 to 14.6 l/min and 1000 to 4800 liters, respectively. These large CL/F and Vd/F values suggest that the oral bioavailability of tea catechins in humans is also low. Because of low oral bioavailability, plasma tea catechin concentrations determined in humans after oral administration of green tea extract or green tea catechins were 5-50 times less than the concentrations shown to exert biological activities in in vitro systems.

Because a significant fraction of the orally administered green tea catechins is not absorbed or is eliminated pre-systemically, small changes in factors limiting the systemic availability of green tea catechins could have a significant impact on their oral bioavailability. It has been shown in this study that taking Polyphenon E on an empty stomach after an overnight fast resulted in a dramatic increase in the blood levels of free EGCG, EGC, and ECG. In addition, taking Polyphenon E under fasting conditions resulted in a decrease in the blood and urine levels of the glucuronide and/or sulfate conjugates of EGC and EC. Formation of glucuronide and sulfate metabolites of pharmaceutical drugs such as acetaminophen has been shown to be reduced in rodents after an acute fast. It has been postulated that an acute fast depletes precursors for the conjugation reactions. It is plausible that less green tea catechins undergo pre-systemic glucuronidation and/or sulfation reactions in the fasting condition, resulting in more free catechins escaping the pre-systemic loss and being available in the systemic blood.

In addition, green tea catechins have been shown to be stable in acidic conditions, but degrade rapidly at pH levels above 7. Thus, fasting after the consumption of a tea polyphenol is thought to improve bioavailability as the presence of food will raise the gastric pH and delay the gastric emptying rate, while the stomach pH ranges from 1 to 3 under the fasting condition. In other words, based on this physiological change, it is expected that green tea catechins would be more stable in the fasted stomach and ultimately has greater oral bioavailability in this dosing condition.

Food has also been shown to decrease the intestinal absorption of pharmaceutical drugs through irreversible interactions between drugs and dietary components or reversible interactions but exhibiting an absorption window in the proximal small intestine, a decrease in drug dissolution rate as a result of elevation of the luminal viscosity, and interactions between drugs and bile acids secreted following food ingestion. These factors could also contribute to the dramatic increase in the oral bioavailability of green tea catechins observed in the fasting condition.

It is not known whether taking Polyphenon E with a meal with compositions different from that used in our study would provide better subject tolerance at higher doses and not impair the oral bioavailability of green tea catechins. A recent study has assessed the impact of different macronutrients on flavanol (EC+catechin) absorption from sugar free, flavanol-rich cocoa. It was found that flavanol absorption is increased significantly by concurrent consumption of carbohydrate rich meals, including sugar, bread, and grapefruit juice (˜140% increase in AUC values). Lipid and protein rich meals such as butter, milk, and steak had minimal effects on flavanol absorption. The muffins provided in our study are rich in carbohydrate and fat content. Since carbohydrate rich meals already provided facilitated favanol absorption when compared with lipid and protein rich meals, it is expected that lipid and protein rich meals would also reduce the oral bioavailability of green tea catechins in comparison with the fasting condition.

All doses of Polyphenon E used in this trial were generally well tolerated after single dose administration. Mild and transient nausea was noted in some of the study participants and was seen most often at the highest study agent dose (1200 mg EGCG) and in the fasting condition. The study shows that taking Polyphenon E at doses that contain 400 or 800 mg EGCG in the fasting condition is as well tolerated as taking Polyphenon E with food.

In summary, greater oral bioavailability of free catechins can be achieved by taking Polyphenon E on an empty stomach after an overnight fast, i.e., between 6 hours and 12 hours. This dosing condition is also expected to maximize polyphenol biological effects. For chronic use, the recommended maximum tolerated dose of Polyphenon E for this dosing condition is a dose that contains 800 mg EGCG.

Various changes in the details and components that have been described may be made by those skilled in the art within the principles and scope of the invention herein described in the specification and defined in the appended claims. Therefore, while the present invention has been shown and described herein in what is believed to be the most practical and preferred embodiments, it is recognized that departures can be made therefrom within the scope of the invention, which is not to be limited to the details disclosed herein but is to be accorded the full scope of the claims so as to embrace any and all equivalent processes and products.

What is claimed is:

1. A method for administering a tea polyphenol, comprising the step of providing instructions to a user to fast for a defined period prior to tea polyphenol intake.

2. The method of claim 1, wherein said instructions are in printed or electronic form.

3. The method of claim 1, wherein said defined period is between 6 and 12 hours.

4. The method of claim 1, wherein said defined period is between 8 and 10 hours.

5. The method of claim 1, wherein said tea polyphenol comprises Polyphenon E.

6. The method of claim 1, further comprising the step of fasting after tea polyphenol intake for at least 5 minutes.

7. A method for increasing catechins in blood serum, comprising the steps of:
a. fasting for a defined period; and
b. consuming a tea polyphenol after said defined period.

8. The method of claim 7, wherein said catechins increase in concentration in blood serum by at least two fold.

9. The method of claim 7, wherein said defined period is between 6 and 12 hours.

10. The method of claim 7, wherein said defined period is between 8 and 10 hours.

11. The method of claim 7, wherein said tea polyphenol comprises Polyphenon E.

12. The method of claim 7, further comprising the step of fasting after tea polyphenol consumption for at least 5 minutes.

13. A method for enhancing chemoprevention, comprising the step of a user consuming a tea polyphenol after fasting for at least 6 hours.

14. The method of claim 13, wherein said user's blood serum concentration of a catechin is increased by at least two fold.

15. The method of claim 13, further comprising the step of fasting after tea polyphenol intake for at least 5 minutes.

16. The method of claim 13, wherein said fasting is between 6 and 12 hours.

17. The method of claim 13, wherein said fasting is between 8 and 10 hours.

18. The method of claim 13, wherein said tea polyphenol comprises Polyphenon E.

19. The method of claim 18, wherein 800 milligrams or less of said Polyphenon E is consumed.

20. The method of claim 13, wherein the user receives printed or electronic instructions to fast for at least 6 hours prior to tea polyphenol intake.
